(19) United States
(12) Patent Application Publication
(10) Pub. No.: US 2019/0381094 A1
(43) Pub. Date: Dec. 19, 2019

(54) Biochloride Generation and Methods

(71) Applicant: MCP IP, LLC, Dover, DE (US)

(72) Inventors: Marcy C. Purnell, Hernando, MS (US); Matthew B.A. Butawan, Memphis, TN (US)

(21) Appl. No.: 16/478,883

(22) PCT Filed: Jan. 18, 2018
(86) PCT No.: PCT/US18/14238

§ 371 Date: Jul. 18, 2019

Related U.S. Application Data
(60) Provisional application No. 62/447,704, filed on Jan. 18, 2017.

(51) Int. Cl. A61K 33/20; C01B 7/01; C12N 13/00

(52) U.S. Cl. CPC A61K 33/20; C12N 13/00; C01B 7/01

(57) ABSTRACT

The method of enhancing diamagnetic anisotropy in cell membranes that occur under the influence of disassociated polymorphic _{b}Cl⁻ comprising changes in chloride ion channel expression and cell physiology. Direct current from a device generates an electromagnetic field in a 3 hypotonic saline solution leading to a dielectrophoretic disassociation of the chloride ion from its chloro-metabolites transforming it into a polymorphic diamagnetically disassociated bio-chloride (_{b}Cl⁻). This field treated aqueous solution induces a magnetic moment change in solution for some hours when no longer under the influence of the direct current; for when this field influenced solution is used to reconstitute growth media of human breast carcinoma (MDA-MB-231) and human breast epithelial (MCF-10A) cells in vitro, significant changes in chloride ion channel expression, membrane potential, cell volume, and a massive transcriptional reprogramming of 2,468 genes expressions by Human Genome U133 Plus 2.0 Gene Chip Array (Affymetrix) analyses occur.

### CROSS-REFERENCE TO RELATED APPLICATIONS

This application claims priority to U.S. Provisional Application No. 62/447,704 filed on Jan. 18, 2017, the contents of which are incorporated herein by reference in its entirety.

### FIELD OF THE INVENTION

This invention is generally directed to methods of generating biochloride and applications thereof. More specifically, the invention is related to methods of modulating chloride ions and ions channels.

### BACKGROUND OF THE INVENTION

Chloride channels are ubiquitously expressed in plasma membranes and intracellular organelles and are known to play an important role in many cellular processes such as the regulation of intracellular pH, membrane potential (Vmem), transport across membranes, and cell volume homeostasis (Cummings et al., 2016). Chloride channels are antiporters/cotransporters and integral membrane proteins that carry out secondary transport of two or more different molecules or ions bi-directionally, across a phospholipid membrane (Duran et al., 2010). A recent discovery also shows that chloride ion signaling plays a key role in the formation of the basement membrane that exists outside of the cells and also guides many functions in all cells (Peretti et al., 2015). Chloride ions form hydrogen-bonded bridges with water molecules which differ from the orientation of other ions (sodium and potassium) that instead bond to the oxygen side of water (Mancinelli et al., 2007). Chloride ions are also considered to be a kind of second messenger in cells as they bind to many proteins that interface with intracellular organelles and membrane channel transporters (Duran et al., 2010). To date, science has not been able to generate the ‘switch’ that modulates conformational changes in the chloride ion that are ultimately needed to enhance and affect chloride ion channel function and influence cell physiology in living organisms (Peretti et al., 2015; Tseng and Levin, 2013).

In recent years, dielectrophoresis (DEP) has been studied due to its potential to influence microparticles, nanoparticles, and cells (Wissner-Gross, 2013; Pommer et al., 2008). Since cells are diamagnetically reoriented by magnetic fields, applications of DEP are currently being pursued in the fields of medical diagnostics, drug discovery, cellular therapeutics, and particle filtration (Iwasaka, 2008; Conroy et al., 2008). DEP can be defined as the net force experienced by a dielectric (able to be polarized) particle in an electric field (Lungu et al., 2011). This force does not require the particle itself to be charged and all particles exhibit dielectrophoretic activity in the presence of electric fields. However, the strength of the force depends on the medium, the electrical properties of the particles, the particle's shape and size, as well as the designed frequency of the field. Therefore, fields can be designed to influence and modulate particles with great selectivity (Lungu et al., 2011). The DEP force (F_{DEP}) can be written where E is the electric field, ∇ is the divergence of the dipole moments, and m (ω) is the induced dipole moment on the particle as in the following equation:

F_{DEP}=(m(ω)−∇)E

DEP can occur when a polarizable particle (ion) is suspended in a field that is driven by alternating current (AC) or direct current (DC). Therefore, DEP can be used to separate particles and sort cells according to their dielectric properties and sizes (Iwasaka et al., 2008). When a particle is more polarizable than the surrounding medium the net movement of the particle is oriented towards the regions of the highest field strength or positive dielectrophoresis (pDEP); whereas particles with polarizability less than that of the medium move towards the region of the lowest field gradient or negative dielectrophoresis (nDEP). Therefore, diamagnetic particles of chloride and water will orient in an opposing direction of the positively charged particles (cations) of sodium (paramagnetic), molybdenum (paramagnetic), nickel (ferromagnetic), chromium (anti-ferromagnetic), etc., leading to particle, cell, and/or cell tissue separations (Iwasaka et al., 2008). These dissociations or separations of nanoparticles through DEP offer potential for these charged particles to change their magnetic behavior and function as is seen with the application of the carefully designed DC-DEP force electromagnetic field (EMF) frequency generated by the Bio-Electric Field Enhancement (BEFE) Device (FIG. 1). This BEFE device is used to apply the frequency in this research and was invented by Terence J. Skrinjar in 1996.

### BRIEF DESCRIPTION OF THE DRAWINGS

Further advantages of the invention will become apparent by reference to the detailed description of preferred embodiments when considered in conjunction with the drawings:

FIG. 1 depicts the BEFE device producing a specifically designed DEP force frequency from a DC initiated EMF leading to the dissociation of chloride ions from their chloro-metabolites in a 3 mM hypotonic saline solution (5 M NaCl and deionized water).

FIG. 2A-2D depict a line graphs showing selective growth inhibition of only cancerous cells in human breast carcinoma (MDA-MB-231) and human breast epithelial (MCF-10A) cells in media that had been reconstituted with a hypotonic saline solution that was treated/exposed to the BEFE device generated DC-DEP force EMF or not treated/exposed to the DC-DEP force EMF prior to reconstitution. Individually, FIG. 2A shows MDA-MB-231 cells treated vs. the control. FIG. 2B shows MCF-10A cells treated vs. the control. FIG. 2C shows the autoclaving of the hypotonic saline solution prior to reconstitution shows that exposure to the heat nullified the growth inhibitory effect on cancerous cells, which shows that a magnetic moment factor could be involved in these results. FIG. 2D shows MDA-Media Treated on Day 1 only and changed daily with Treated on Day 1 media vs. the control wherein the applied field continues to exert DC-DEP force EMF in solution before waning after approximately 90 hours as seen in the growth inhibitory effects.

FIG. 3A-3B depict line graphs showing experiments with only the addition of metal salts and no exposure to the DC-DEP force EMF showed no significant growth differences between the cancerous the noncancerous treated and control groups. Individually, FIG. 3A shows MDA-MD-231 reconstituted with “metal” salts vs. the control. FIG. 3B shows MCF-10A reconstituted with “metal” salts vs. the control.

FIG. 4 depicts re-orientation of the ions in solution allowing the negatively charged diamagnetic chloride ions to exhibit both a strong attraction to the diamagnetic hydrophilic properties in the filter membrane and an opposing repulsion to help facilitate movement of the positively charged cations through the 0.45 micron pore filter membrane.

FIG. 5A-5B depict tubulin staining of the MDA-MB-231 cells which showed that approximately 17 percent of the control cells were in different stages of mitosis while the no treated cells were found to be undergoing mitosis after one day of growth in the treated media.

FIG. 6A-6B depict bar graphs showing MDA-MB231 treated groups showed an increase in cell size on days 1-5 when compared to control and then appear to have a drop in cell size on day 6. Individually, FIG. 6A shows MDA-MB-231 cells, and FIG. 6B shows MCF-10A cells.

FIG. 7A-7B depict bar graphs showing MCF-10A cells measured larger than the MDA-MB231 cells when initially placed in the treated and control media, but overall kept uniform size between control and treated groups from day 1-6. Individually, FIG. 7A shows MDA-MB-231 cells, and FIG. 7B shows MCF-10A cells.

FIG. 8A-8B depicts line graphs showing hyperpolarization of both cell lines initially observed when exposed to treated media. Individually, FIG. 8A shows MDA-MB-231 cells and FIG. 8B shows MCF-10A cells.

FIG. 9A-9B depict changes in the morphology and size of the cancer cells in phase contrast pictures after one day in treated and control growth media.

FIG. 10 depicts a bar graph showing a significant increase in mRNA in the treated versus control groups in the human breast carcinoma which shows that an increase in transcription or mRNA stabilization is occurring in the treated versus control groups.

FIG. 11 depicts a diagram showing another significant up-regulation in the serine and glycine biosynthesis pathways in both the human breast carcinoma and the human breast epithelial cells.

FIG. 12A-12B depicts a representation of the Golden Ratio that could be used to measure the optimal proportions that are required for the optimal efficiency of the human erythrocyte. Individually, FIG. 12A depicts geometric proportions of the Golden Ratio of a red blood cell. FIG. 12B depicts the golden ratio superimposed over Band3/AE1 channels.

FIG. 13A-13B depicts chloride residing immediately adjacent to the surface membrane on the red blood cell since it is the gatekeeper of Band3/AE1 and separation between the negative surface membrane (where the chloride resides) and the positively charge cationic Stern layer is essential to maintain a strong zeta potential or a static current flow on the surface of the torus/red blood cell. Individually, FIG. 13A shows the non-polarized capacitance red blood cell, and FIG. 13B shows polarized capacitance red blood cells.

FIG. 14A-14C depicts BFA's dc DEP EMF appears to enhance zeta potential thereby ensuring the appropriate geometric proportions that form Golden Ratio of the red blood cell and that are necessary for the electromagnetic excitation driven toroidal multipole flow. Individually, FIG. 14B shows that BGA's dc DEP EMF ultimately maintains the efficiency of the center dielectrophoretic field.

FIG. 15A-15B depicts light microscopy of live blood analysis. Individually, FIG. 15A shows the live blood before treatment with the BFA, and FIG. 15B shows the live blood after treatment with BGA. Note the Red Blood Cell returns to the geometric proportions of the Golden Ratio (1.618).

### SUMMARY OF THE INVENTION

We disclose a method of directing current from a device that generates an electromagnetic field in a hypotonic saline solution leads to a dielectrophoretic disassociation of the chloride ion from its chloro-metabolites transforming it into a polymorphic diamagnetically disassociated bio-chloride (bCl—). This field treated aqueous solution appears to continue to induce a magnetic moment change in solution for some hours when no longer under the influence of the direct current; for when this field influenced solution is used to reconstitute growth media of human breast carcinoma (MDA-MB-231) and human breast epithelial (MCF-10A) cells in vitro, significant changes in chloride ion channel expression, membrane potential, cell volume, and a massive transcriptional reprogramming of 2,468 genes expressions by Human Genome U133 Plus 2.0 Gene Chip Array (Affymetrix) analyses occur. Strong changes in chloride ion channel expression and cell physiology are linked to enhanced diamagnetic anisotropy in cell membranes that occur under the influence of this disassociated polymorphic bCl—.

We further disclose herein the method of alter the magnetic behavior of the chloride ion in all cell membranes with The BFA-dc-DEP-EMF. BFA-dc-DEP-EMF modulates the zeta potential through ferromagnetic, ferroelectric and ferroelastic changes in order to restore the designed Golden Ratio of the red blood cell and offer a new way to treat red blood cell rheology and ultimately cell, tissue and organism pathology with a novel dielectrophoretic electromagnetic application

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Chloride channels represent ubiquitously expressed proteins that regulate fundamental cellular processes including membrane potential, maintenance of intracellular pH, and regulation of cell volume. However, mechanisms to modulate this large family of ion channels have remained elusive to date. This large chloride channel family does not appear to operate with selectivity similar to the sodium and potassium channels. These unique channels appear to be bi-directional cotransporters of two or more different molecules or ions across a bilayer phospholipid membrane.

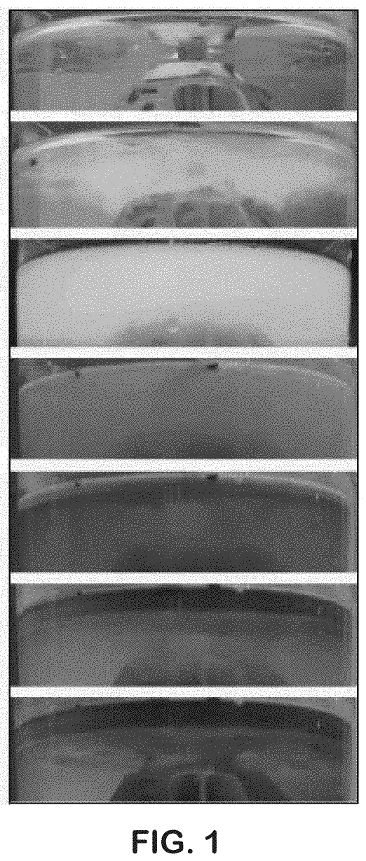

The BEFE device produces a specifically designed DEP force frequency from a DC initiated EMF that leads to the dissociation of chloride ions from their chloro-metabolites in a 3 mM hypotonic saline solution (5 M NaCl and deionized water), as shown in FIG. 1. This device is driven by 3 amperes of DC that is directed through an array that consists of a series of conductive rings and is placed in this hypotonic saline solution. Living organisms such as cells, plants, animals, and humans that come in contact with this field exposed solution show interesting effects. This device has been used across the globe for 20 years as a bath/footbath with anecdotal reports of cancer treatment recovery, improved wound healing, pain relief, improvements in Parkinson's disease and autism, etc. (Marsh, 2001). In vitro studies were conducted to see if any significant effects could be quantified with scientific rigor. These studies showed that when the chloride ions in this hypotonic saline solution are exposed to the DC-DEP force EMF from this device, a polymorphic diamagnetic molecule termed bio-chloride (_{b}Cl⁻) is created when this molecule disassociates from other chloro-metabolites in solution (Mancinelli et al., 2007). This _{b}Cl⁻ was named for its ability to exhibit magnetic behavioral and possibly polymorphic structural changes that allow it to modulate biological functions such as cell growth, chloride channel ion channel expression, membrane potential, cell volume, and massive transcriptional reprogramming in human breast carcinoma (MDA-MB231) and human breast epithelial (MCF-10A) cells in vitro.

### Experimental Design and Methods

### Cells and Cell Culture Media

Human MDA-MB-231 triple-negative breast carcinoma and human MCF-10A breast epithelial cells were obtained from the American Type Culture Collection. The MDA-MB-231 cells were maintained in high glucose Dulbecco's Modified Eagles Medium (DMEM, Lonza, Allendale, N.J.) containing 10% fetal bovine serum (FBS; Atlanta Biologicals, Flowery Branch, Ga.). MCF-10A cells were maintained in DMEM/F12 (Invitrogen, Carlsbad, Calif.) supplemented with 20 ng/ml epidermal growth factor (PeproTech, New Orleans, La.), 0.5 mg/ml hydrocortisone (Sigma-Aldrich, St. Louis, Mo.), 100 ng/ml cholera toxin (Sigma-Aldrich), 10 μg/ml insulin (Sigma-Aldrich) and 5% horse serum (Invitrogen).

To prepare treated and control DMEM, 10×DMEM (Sigma-Aldrich) was diluted 9:1 with a hypotonic saline solution that had been treated for 30 minutes at 3 amperes of DC with the DC-DEP-EMF device, or with an aliquot of the same solution prior to treatment with the device. The hypotonic saline solution consisted of 3 mM NaCl prepared using laboratory-grade deionized water and molecular biology-grade NaCl (Promega, Madison, Wis.). Complete treated and control DMEM was supplemented with 0.004 gm/L folic acid (Sigma-Aldrich), 4,000 mg/L glucose (Sigma-Aldrich), 0.584 gm/L glutamine (Sigma-Aldrich), and 3.7 gm/L sodium bicarbonate (BioWhittaker, Walkersville, Md.) and filtered through a 0.45 micron pore size bottle top filter (Corning, N.Y., N.Y.). Fetal bovine serum was then added to 10% final concentration.

To prepare treated and control media for growth of the MCF-10A cells, F-12 nutrient mix powder (Life Technologies, Carlsbad, Calif.) was re-suspended in either DC-DEP-EMF-treated saline or with an aliquot of the same solution prior to treatment with the device. The F-12 media was then mixed 1:1 with DMEM prepared as described above with either DC-DEP-EMF-treated or control saline and then the DMEM/F-12 was supplemented with EGF, cholera toxin, and insulin as described above and then filter sterilized as described above. Horse serum was then added to 5% final concentration.

### Cell Growth Studies

Treated groups of MDA-MB231 cells in the DMEM-10 were cultured with media that was reconstituted with a hypotonic saline solution that had been treated with the DC-DEP force EMF for 30 minutes and the control groups were cultured in media that was reconstituted with the same hypotonic saline solution prior to treatment with the BEFE unit. The treated group of MCF-10A cells was cultured in the DMEM/F12-5 media that had been reconstituted with a hypotonic saline solution that had been treated with the DC-DEP force EMF for 30 minutes and the control group was cultured in media that was reconstituted with a hypotonic saline solution that had not been treated with the DC-DEP force EMF. On day one, aliquots of 10,000 cells were plated in three 6-well plates for each of the two groups for each of the two cell lines. They were plated in their standard (non-EMF treated) DMEM-10 or DMEM/F12-5 media on day 1. On day 2, the treated (n=21) and control (n=21) media for each of the two cell lines were made and the original standard media was replaced in each of the wells with the newly prepared treated and control media. On days 3 through 7, media was prepared and changed daily and wells from the control group and treated group of each cell lines were trypsinized, removed from 3 wells of each group and counted and cell size of each sample was measured using a Scepter cell counter (EMD Millipore, Darmstadt, Germany). Another variation of cell growth was also examined. The media was prepared and made only on day 1 and enough was made to use for daily media replacements. Growth data in the growth studies was analyzed with Mann Whitney U if the tests for normality were not met and Student's unpaired two-tailed t-tests if tests for normality were met.

### Membrane Potential Analyses

Membrane Potential analyses were performed on the two cell lines using Five-Photon Membrane Potential Assay (Five-Photon Biochemical, San Diego, Calif.). This assay utilizes an oxonol membrane permeant dye. Vmem assay dyes enter depolarized cells and bind to their intracellular membranes or proteins leading to increased fluorescence. An increase in depolarization leads to an elevated influx of the voltage sensitive dye and an increase fluorescence that can be measured by fluorescent microplate readers or flow cytometers. After washing twice with PBS to remove serum factors, 60,000 cells were plated in the wells of a 96-well plate and placed in 100 mcl of serum-free media. The wells were plated with both the treated and control groups of each cell line. The cells were exposed to the treated and control serum-free media for the time points of: 45 minutes, 8 hours, and 24 hours prior to the addition of 100 mcl of External Assay Buffer. The cells were then incubated in the dark at 37° C. in a CO₂ incubator for 20 minutes to load the dye prior to placing in a fluorescent plate reader. The fluorescence was measured in the 530 excitation\wavelength (nm) and 565 emission wavelength (nm) with a 550 emission cut-off (nm). The lipophilic, anionic dye partitions across the plasma membrane of live cells and is dependent on the Vmem across the membrane. When the cells are depolarized, more indicator dye enters the cells causing an increase in fluorescence signal. These slowly responding oxonol dyes have been known to show changes in fluorescent signal above 80%. Oxonol dyes contain a negative charge which makes them permeant to cell membranes (Brauner, 1984). This method of membrane potential analysis seems to be the least damaging method for determination of Vmem changes and these negatively charged molecules form complexes with many ionophores that could be affected by DC-DEP force EMF application. Also, fluorescence methods are best suited for high-throughput identification of chloride channel influences (Verkman and Galietta, 2009). Data were analyzed based on percent change in fluorescence between the treated and control groups.

### Microarray Analyses

Replicate 60 mm dishes of either MDA-MB-231 or MCF-10A (5 plates each for growth in treated and control media) were plated in DMEM-10 and in the next day the media were replaced with either treated or control media which was replaced daily with freshly prepared treated or control media for the next two days. On day 4 post-plating (day 3 post-treatment) the cells were removed with trypsin, counted and 3×10⁶ cells from each plate were collected by centrifugation and total RNA was isolated using the RNeasy Mini Kit according to the manufacturer's instructions (Qiagen, Hilden, Germany). RNA concentration was determined and RNA integrity was evaluated using an Agilent 2100 Bioanalyzer (Agilent Technologies, Santa Clara, Calif.) and all RNA integrity number (RIN) values were ≥10. The RNAs from the five biologic replicates from each group were combined and cDNA was generated using Ambion WT amplification kit (ThermoFisher Scientific, Foster City, Calif.) according to the manufacturer's instructions. The samples were subsequently fragmented and labeled using the Affymetrix WT Terminal Labeling kit and then hybridized, together with the probe array controls, onto the Human Genome U133 Plus 2.0 GeneChip Array (Affymetrix, Foster City, Calif.). The array was washed and stained using an Affymetrix Fluidics Station 450, scanned on an Affymetrix GCS3000 7G scanner, and the data were normalized by Robust Multichip Averaging (RMA) using the Affymetrix expression console in order to transform all the arrays to have a common distribution of intensities by removing technical variation from noisy data before analysis. To quintile normalize two or more distributions to each other, both treated and control groups were set to the average (arithmetical mean) of both distributions. Therefore, the highest value in all cases becomes the mean of the highest values; the second highest value becomes the mean of the second highest values, etc. Data were analyzed through the use of Qiagen's Ingenuity Pathway Analysis (IPA, Qiagen).

### RT-qPCR Analyses

In order to validate some of the significant chloride ion channel expressions seen in the microarray analysis on the MDA-MB231 cells, Real-Time qPCR was conducted using LC 480 and UPL probes for TaqMan Master Mix protocol. The primers were designed using universal probe library. Transcriptor First Strand cDNA Synthesis Kit (Roche) was used to make cDNA with the original microarray samples. Then the following reagents were added to the wells in the appropriate measurements according to the protocol in order to make 8 mcl of this master mix for each well used in the 5-dilution factors (in triplicate) of the cDNA: universal library probe (UPL probe, Roche) at 10 μM, LC480 master mix (2X concentration, Roche), mixed left and right primers at 10 μM each in DNase, RNase, and Protease-free water (Corning), and nuclease free water. The five dilutions of cDNA were: undiluted, 1:10, 1:100, 1:1,000, 1:10,000. The 8 mcl of the master mix and 2 mcl of the cDNA were added to wells of a 96 well plate. The plates were centrifuged and activated for 5 minutes at 95° C. Then there was an amplification of 45 thermal cycles using 50° C. for 2 minutes (separation), 95° C. for 10 minutes (initialization) and 95° C. for 15 seconds (denaturation), and 60° C. for 1 minute (annealing), and 70-74° C. for 5 minutes (elongation). The amplicons were then plotted for validation. Data were analyzed with both the delta/delta CT method and with Student's unpaired two-tailed t-tests.

### Water Analysis of Metal Ions

In order to determine the basis for the biological effects induced by the DC-DEP force EMF, the ionic composition of the water was examiner before and after treatments and after filtering of the treated water by performing a water analysis. Eight liters of de-ionized water were placed in a 12 liter foot tub plastic wash basin and 5.5 ml of 5 M NaCl were added from which 500 ml were removed and placed in a clean plastic container to serve as the ‘control’ saline solution (per request of A & L). The DC-DEP force EMF array was then placed in the plastic wash basin and 3.0 amps of current applied for 30 minutes. Next, 500 ml of the ‘treated’ saline solution was removed and placed in a clean plastic container. A separate 500 ml of the ‘treated’ saline solution was run through a 0.45 micron filter and placed in a clean plastic container. The three containers of the control, treated, and treated-filtered saline solution were immediately transported for the water ion analysis to A & L Analytical Laboratories in Memphis, Tenn.

Comparison Water Analysis with Metal Ions

In order to determine if the biological effects induced by the DC-DEP force EMF could be replicated without running DC through the device and by simply adding the metal salts noted in the first water analysis in Table 1, the ionic composition of the water was examiner before and after adding the metal salts to the same concentrations (treated) and after filtering of the metal salt water (treated filtered) (Table 2). Eight liters of de-ionized water were placed in a 12 liter foot tub plastic wash basin and 5.5 ml of 5 M NaCl were added. Then the microgram/liter concentrations of molybdenum, chromium, and nickel (see cell growth with metal salts) were added to the 3 mM hypotonic saline solution. Then 500 ml were removed and placed in a clean plastic container and 500 ml were removed and run through a 0.45 micron filter (Corning) and placed in a clean plastic container. The filtered and unfiltered saline/metal salt solution were immediately transported for the water ion analysis to A & L Analytical Laboratories in Memphis, Tenn. Percent change in the metal ion content between filtered and unfiltered solutions were calculated.

Cell Growth with Metal Salts

MDA-MB231 cells were cultured in the DMEM-10 with media that was reconstituted with a hypotonic saline solution that had chromium, nickel, and molybdenum salts added to the same concentrations as found in the water analysis in Table 1 and the control groups were cultured in media that was reconstituted with the hypotonic saline solution that had not been treated with the DC-DEP force EMF system. The treated group of MCF-10A cells was cultured in the DMEM/F12-5 media that had been reconstituted with a hypotonic saline solution that had chromium (chromium chloride hexa-hydrate, Aldrich), nickel (nickel chloride hexa-hydrate, Sigma), and molybdenum (Aldrich) salts added to the same concentrations as found in the water analysis in Table 1, and the control group was cultured in media that was reconstituted with a hypotonic saline solution that had not been treated with the DC-DEP force EMF. Cell growth protocol was then followed, and data were tested for normality and analyzed with Student's unpaired two-tailed t-tests.

### Results

### Cell Growth and Water Analyses

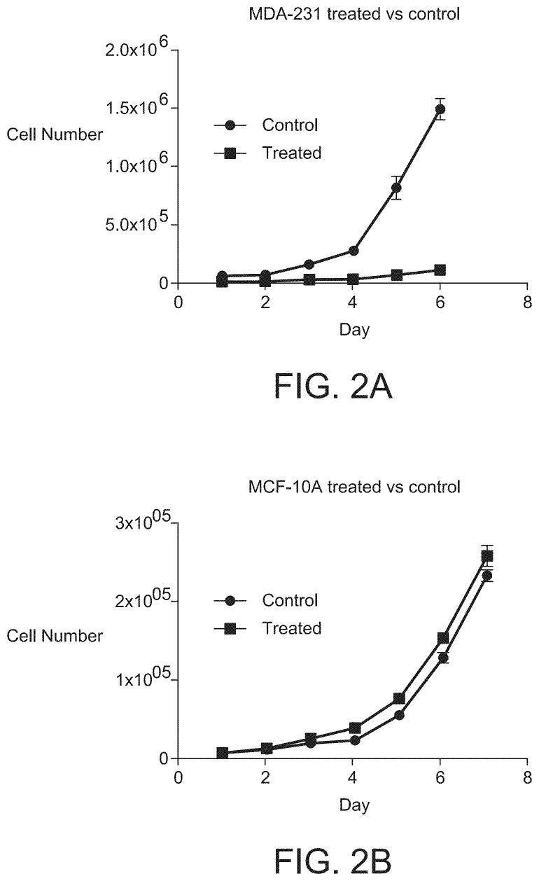

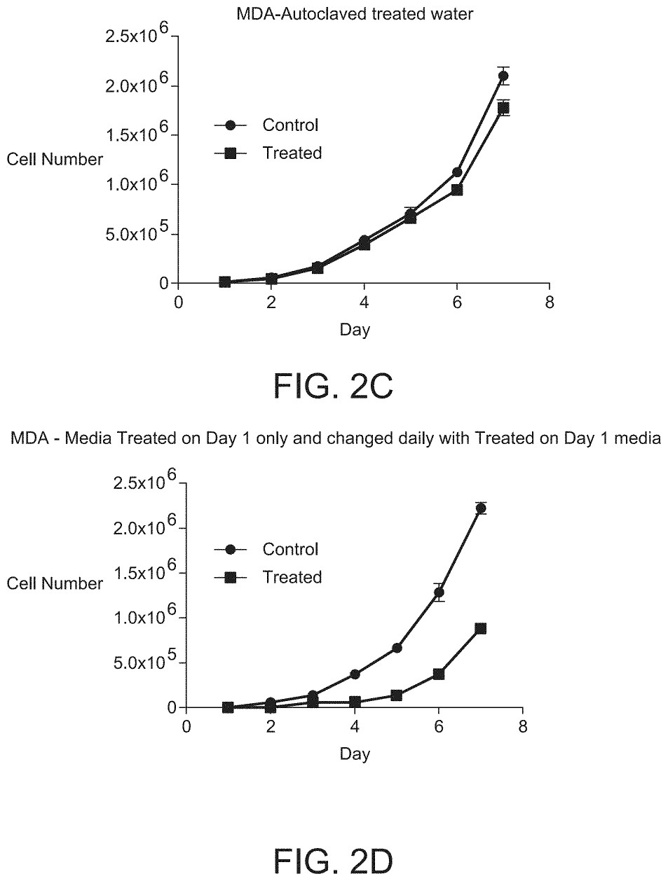

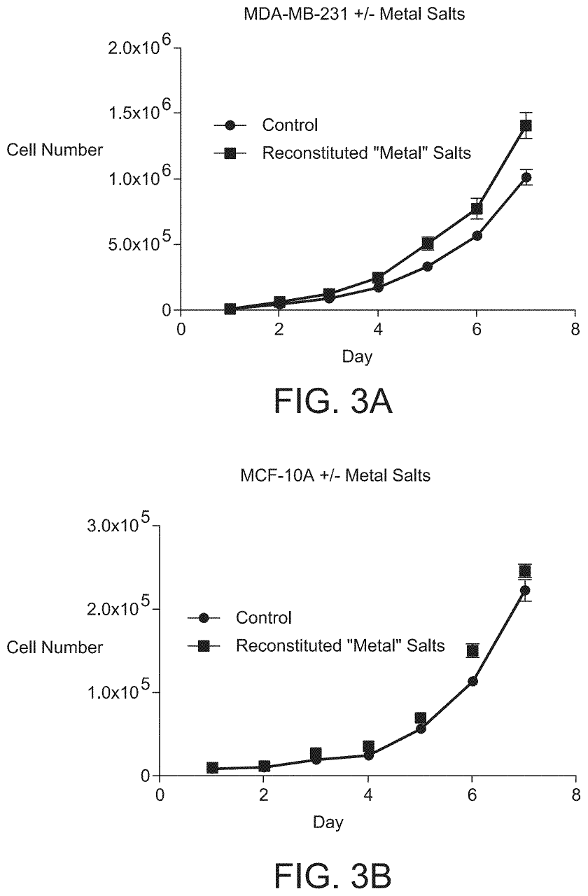

First, differences in growth were assessed between cancerous and noncancerous cells in vitro when grown in the treated and control media. Human breast carcinoma (MDA-MB-231) and human breast epithelial (MCF-10A) cells were cultured in media that had been reconstituted with a hypotonic saline solution that was treated/exposed to the BEFE device generated DC-DEP force EMF or not treated/exposed to the DC-DEP force EMF prior to reconstitution. These initial experiments showed the unexpected finding of selective growth inhibition of only the cancerous cells in these two cell lines, as shown in FIGS. 2A and 2B. In order to determine a mechanism that was occurring in the water to lead to this growth inhibition, a water analysis of the hypotonic saline solution was conducted to see if there were detectable differences in ionic composition between the DC-DEP force EMF treated hypotonic saline solution, the treated filtered solution (needed to conduct in vitro study) and the control/untreated solution (Table 1). To determine if the metal ion differences noted between the control hypotonic solution, treated hypotonic solution, and filtered/treated hypotonic solution could explain these selective breast carcinoma growth inhibitory effects, growth experiments were conducted by making growth media with metal salt additions to achieve the same concentration of metal ions in solution that were measured in independent laboratory analyses of the control and filtered/treated solutions (Table 1). Conducting these experiments with only the addition of metal salts and no exposure to the DC-DEP force EMF showed no significant growth differences between the cancerous the noncancerous treated and control groups, as shown in FIG. 3A-3B. No chloride was measured in the water analysis of the treated/filtered hypotonic saline solution, and, therefore, the chloride in the treated/filtered hypotonic saline solution had completely disappeared with the filtering process (Table 1). Alternatively, filtering of the saline solution in the metal salt experiment did not show a filtering out or removal of the chloride, but rather a 33% increase in chloride concentration over the unfiltered solution (Table 2). Since heat is known to negate dielectric/magnetic properties of ions, treated/unfiltered water was autoclaved at 120° C. and 1.1 bar for 40 minutes prior to reconstitution of the growth media in the next set of experiments (Sverjensky et al., 1997). The autoclaving of the hypotonic saline solution prior to reconstitution shows that exposure to the heat nullified the growth inhibitory effect on cancerous cells, which shows that a magnetic moment factor could be involved in these results, as shown in FIG. 2C. Next, magnetic properties of these metal ions were examined to determine an explanation regarding how the DC-DEP force EMF might be linked to the chloride ions' inability to pass through the 0.45 micron pore size filter while the non-DC DEP force EMF chloride ions continued to pass through the filter (Tables 1 and 2).

TABLE 1
Table 1. Analysis of dc-DEP Force EMF Influence on Metal
Aqueous Ions in Control and Treated (Unfiltered/Filtered) Dilute
Saline (3 mM). Analyzed by A and L Laboratories, Memphis, TN.

| | | | Treated |
|---|---|---|---|
| | Control (Unfiltered) | Treated (Unfiltered) | (Filtered) |
| Ion/Element | Concentration | Concentration | Concentration |
| Chloride | 205 mg/L | 128 mg/L | NR* |
| Chromium | 1.13 μg/L | 22.7 μg/L | 18.8 μg/L |
| Molybdenum | <1 μg/L | 2.58 μg/L | 2.4 μg/L |
| Nickel | <0.5 μg/L | 12.1 μg/L | 7.49 μg/L |
| Sodium | 128 mg/L | 82.8 mg/L | 83.3 mg/L |

*NR—none registered/measured.

Analysis of DC-DEP Force EMF Influence on Metal Aqueous Ions in Control and Treated (Unfiltered/Filtered) Dilute Saline (3 mM). Analyzed by A & L Laboratories, Memphis, Tenn.

TABLE 2
Table 2. Percent Differences of Aqueous Metal Ions in dc-DEF
EMF Treated Unfiltered vs. Filtered and Meal Salt Unfiltered versus
Filtered Solutions. Analyzed by A and L Laboratories, Memphis, TN.

| | | dc-DEP | Metal Salts |
|---|---|---|---|
| | | force EMF Treated | (Unfiltered- |
| Ion/Element | Magnetism | (Unfiltered-Filtered) | Filtered) |
| Chloride | Diamagnetic | ↓100% | ↑33% |
| Chromium | Anti-ferromagnetic | ↓17% | ↓32% |
| Nickel | Ferromagnetic | ↓38% | ↓02% |
| Sodium | Paramagnetic | ↓00% | ↑21% |
| Molybdenum | Paramagnetic | ↓06% | ↓58% |

Percent Differences of Aqueous Metal Ions in DC-DEP EMF Treated Unfiltered vs. Filtered and Metal Salt Unfiltered versus Filtered Solutions. Analyzed by A & L Laboratories, Memphis, Tenn.

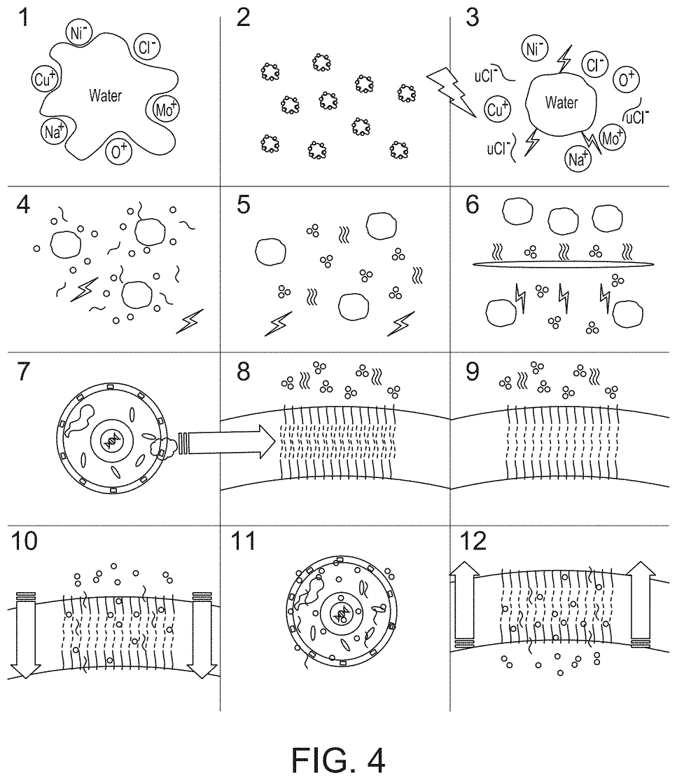

Chloride is a known diamagnetic ion which maintains a negative charge while the other metal ions that were found in the water analysis of the hypotonic saline solution consisted of the positively charged cations: chromium, molybdenum, nickel, and sodium (Table 2). The 0.45 micron pore size filter used in these experiments is composed of a cellulose acetate membrane that carries a low positive static charge and is also hydrophilic in nature (Lonsdale et al., 1965). The acetylation of the cellulose ester acts as a substitute for an active hydrogen (H⁺) atom and appears to attract the DC-DEP force EMF dissociated and negatively charged _{b}Cl⁻. The DC-DEP force EMF from the BEFE device both separates the ions and enhances the opposing orientations of the negatively charged (chloride) and positively charged (sodium, chromium, nickel, and molybdenum) ions in solution, as shown in FIG. 1. This re-orientation of the ions in solution allows the negatively charged diamagnetic chloride ions to exhibit both a strong attraction to the diamagnetic hydrophilic properties in the filter membrane and an opposing repulsion to help facilitate movement of the positively charged cations through the 0.45 micron pore filter membrane, as shown in FIG. 4 (Lonsdale et al., 1965). This analysis of the filtering process also indicates how this DC-DEP force EMF leads to a magnetic modulation of ion flow through separation/attraction/repulsion forces in cell membranes. The separation of all ions occurs first and then the ions will re-orient to those ions to which they are more similar, and, therefore, more strongly attracted to (diamagnetic-chloride ions attract to each other and to diamagnetic-water etc.). Then, the ions transition to opposition/repulsion of less similar ions (paramagnetic-sodium ions oppose diamagnetic-chloride ions etc.) and a separation of molecules, cells, tissues, etc., occurs, as depicted in FIG. 4. Also as previously noted, when the ion concentrations of the unfiltered and filtered hypotonic saline solutions that were used for the metal salt experiments were tested, it was determined that the static charge on the cellulose acetate hydrophilic membrane did not attract the chloride ions that had not been exposed to the DC-DEP force EMF since they readily passed through the 0.45 micron pore filter as shown in FIG. 4 and Table 2. This DC-DEP force EMF driven separation/attraction/repulsion of ions and its associated chloride ion behavioral changes could be strong modulators of diamagnetic anisotropy in the cell membranes.

This data also shows that exposure of the hypotonic saline solution to this DC-DEP force EMF creates a form of magnetic memory change or field effect in solution as well since the chloride ion completely filters out of the solution prior to reconstitution of growth media. It was next determined if this _{b}Cl⁻ completely filters out in the cellulose membrane, then how is this effect translated to these in vitro experiments? Sodium and molybdenum are paramagnetic metal ions, and when these ions are exposed to an electromagnetic field, they form induced magnetic fields in the same direction as the field (Stohr et al., 2006). Chromium is an anti-ferromagnetic metal ion, and it forms an anti-parallel spin that strengthens the field when in combination with parallel spin of the ferromagnetic element nickel (Camley et al., 1989). Ferromagnetic metals (nickel) are highly susceptible to magnetic fields and retain their magnetism even after the removal of the applied field, while anti-ferromagnetic metal ions tend to change their magnetic behaviors relative to the Neel temperature (Ohno et al., 2000). Since nickel is a ferromagnetic metal ion and is found in the treated/filtered hypotonic saline solution, the continued magnetism and/or spin state will continue to influence the other ions in the media components after reconstitution. It is known that diamagnetic metals are magnetized 180° opposite of the applied field and actually repel the applied field when compared to paramagnetic and ferromagnetic metals that are magnetized in the same direction of the applied field (Lungu et al., 2011). Since the strength of the DEP force depends on: the medium (hypotonic saline), the electrical properties of the particles (diamagnetic—water and chloride; paramagnetic—sodium and molybdenum; anti-ferromagnetic-chromium and/or ferromagnetic-nickel), the particles size (Cl⁻ 184 r (pm), Na⁺ 190 r (pm), Ni⁺ 149 r (pm), Cr⁺ 166 r (pm), Mo⁺ 190 r (pm), as well as the frequency of the field, each of these ions will display individual separation/attraction/repulsion behaviors to establish their own signature ‘space’ in solution after exposure to the DC-DEP force EMF, as shown in FIGS. 1 & 4. The discussion below focuses on the DC-DEP force EMF effects on membrane potential, cell volume, tubulin staining, transcription, translation and chloride ion channel expression.

### Tubulin Staining, Cell Volume, Membrane Potential

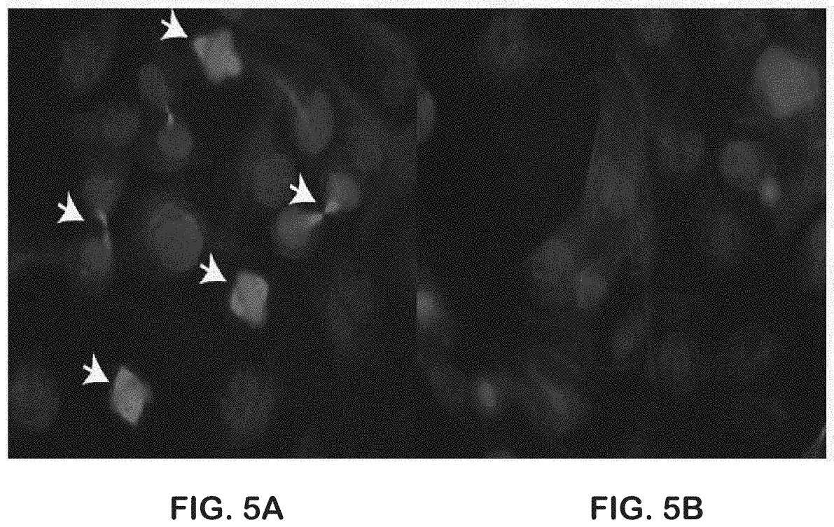

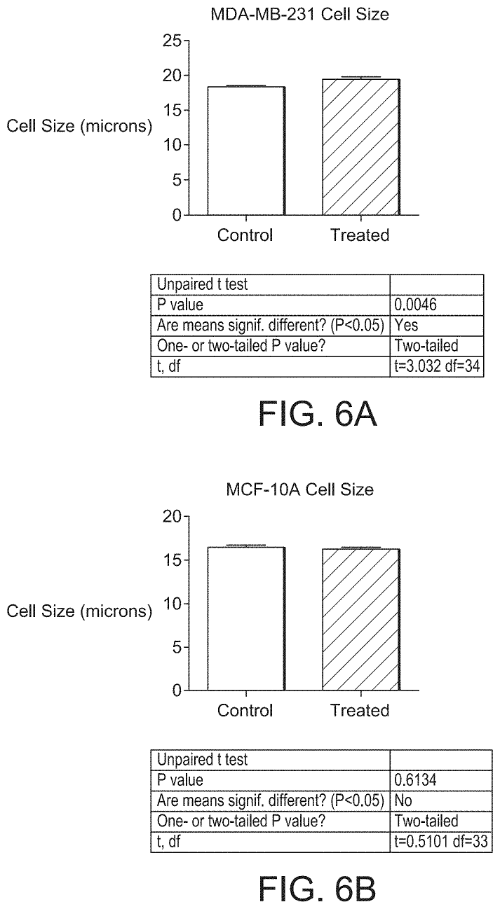

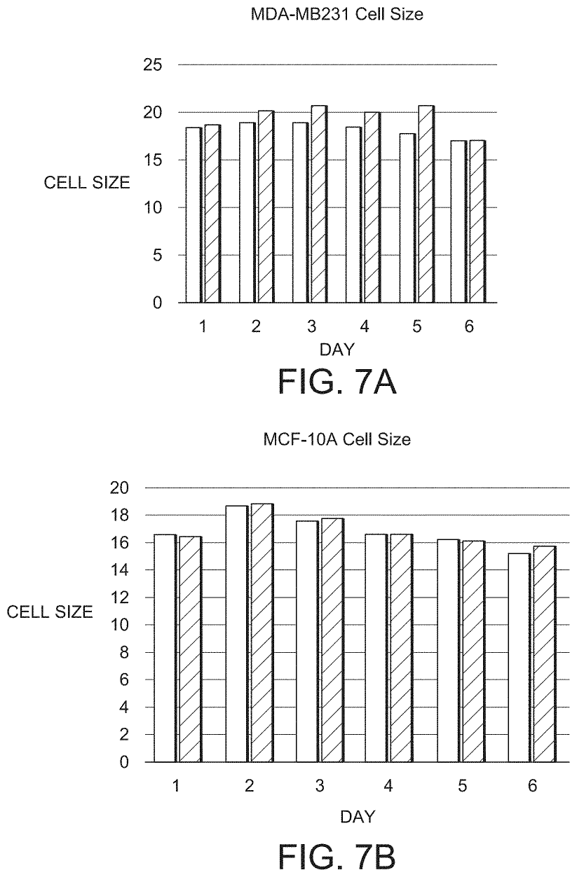

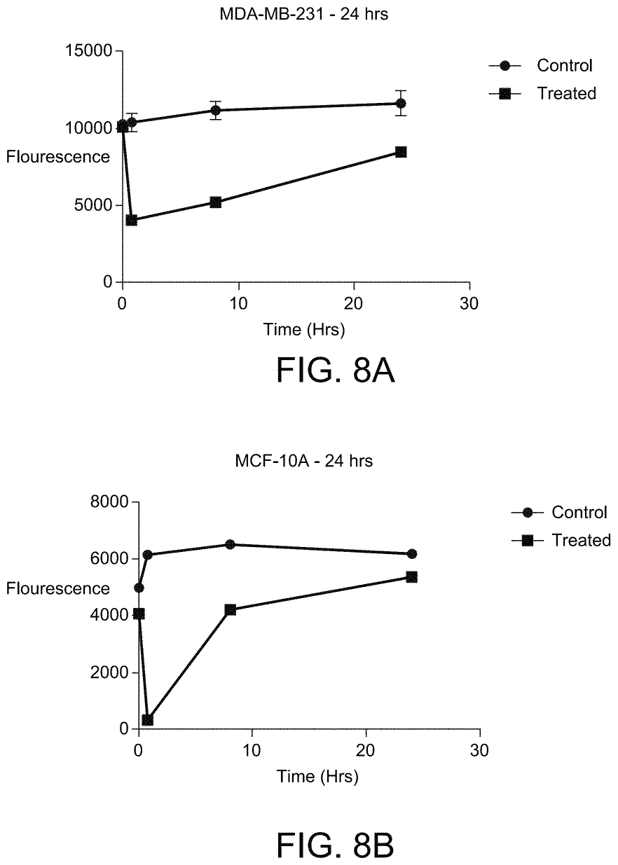

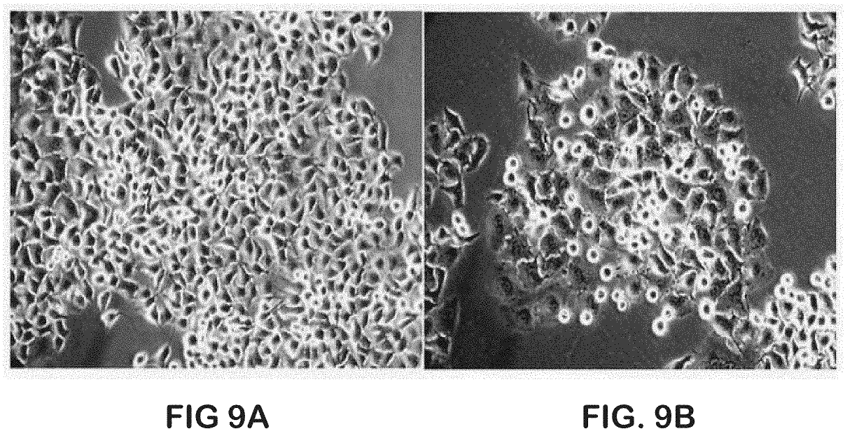

Since chloride ions are known to bind to actin and tubulin, tubulin staining of the MDA-MB-231 cells was conducted which showed that approximately 17 percent of the control cells were in different stages of mitosis while the no treated cells were found to be undergoing mitosis after one day of growth in the treated media, as shown in FIG. 5A-5B. (Suh, 2012; Peretti et al., 2015). Cell volume/size is known to be controlled by chloride ion channels and these were measured daily while conducting the cell growth experiments (Peretti et al., 2015). The MDA-MB231 treated groups showed an increase in cell size on days 1-5 when compared to control and then appear to have a drop in cell size on day 6, as shown in FIG. 6. The MCF-10A cells measured larger than the MDA-M B231 cells when initially placed in the treated and control media, but overall kept uniform size between control and treated groups from day 1-6, as shown in FIG. 7A-7B. Chloride ion channels are also known to be regulators of Vmem. Since fluorescence methods are best suited for high-throughput identification of chloride-channel modulators, membrane potential analysis was conducted with a fluorescent assay (Verkman and Galietta, 2009). Hyperpolarization of both cell lines was initially observed when exposed to treated media, as shown in FIG. 8A-8B. In the first 45 minutes, the MDA-MB231 cells showed a 70% increase in the treated versus control groups while the MCF-10A increased 94%. At the 8 hour measurement there remained a 61% increase in the MDA-MB231 and a 31% increase in the MCF-10A cells (Table 3). At 24 hours the MDA-MB-231 retained a 32% increase while the MCF-10A have returned closer to baseline with a 12% increase (Table 3). Changes in the morphology and size of the cancer cells is also noted from phase contrast pictures after one day in treated and control growth media, as shown in FIG. 9A-9B.

TABLE 3
Table 3. Percent Change in Vmem of Treated vs.
Control Groups of MDA-MB231 and MCF-10A cells.

| | | 8 Hours in | 24 Hours in |
|---|---|---|---|
| | 45 Minutes in Treated | Treated Media | Treated Media |
| | Media as Compared to | as Compared | as Compared |
| Cell Line | Controls | to Controls | to Controls |
| MDA-MB231 | ↑70% | ↑61% | ↑32% |
| MCF-10A | ↑94% | ↑31% | ↑12% |

Percent Change in Vmem of Treated vs. Control Groups of MDA-MB231 and MCF-10A cells.

### Microarray and RTqPCR Analyses

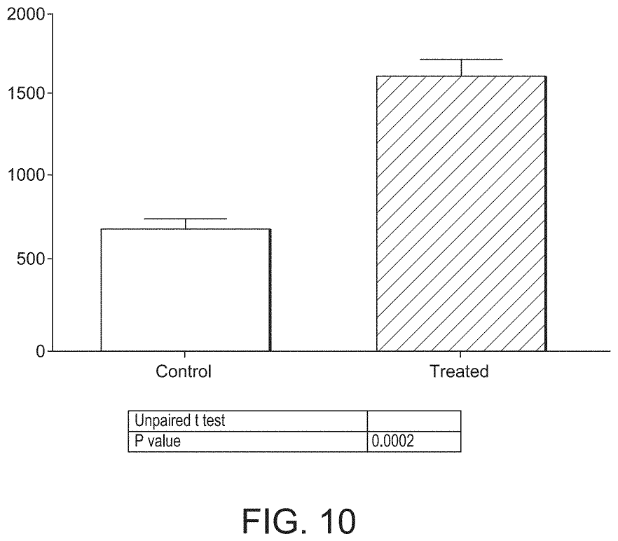

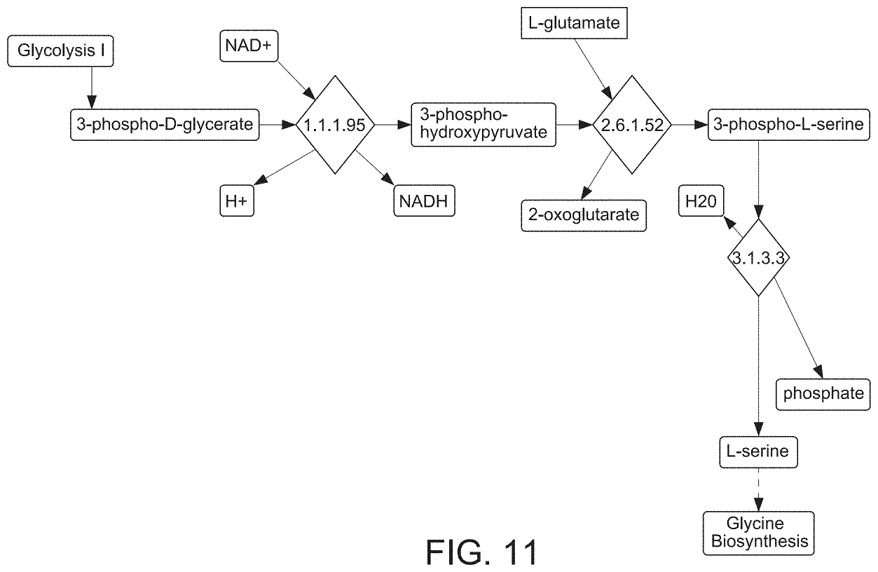

Microarray analysis identified 1,165 genes that were up-regulated over 2-fold and 872 genes that were down-regulated over 2-fold in the treated groups grown in the media that was reconstituted with the DC-DEP force EMF-treated hypotonic saline solution compared to the non-treated human breast carcinoma. For comparison, 431 transcripts showed a significant increase in mRNA in the treated versus control groups in the human breast carcinoma which shows that an increase in transcription or mRNA stabilization is occurring in the treated versus control groups, as shown in FIG. 10. Another significant up-regulation was noted in the serine and glycine biosynthesis pathways in both the human breast carcinoma and the human breast epithelial cells, as shown in FIG. 11. This up-regulation shows that the cancerous and noncancerous cells are showing an increase glycine synthesis and possibly amino acid/nucleic synthesis. It is interesting to note that glycine receptors and chloride channels are intricately linked (Lynch, 2004). A t-RNA up-regulation of L-cysteine, L-glutamine, L-glutamate, L-alanine, L-tyrosine, L-tryptophan, L-serine, L-proline, and L-glycine were also found in the human breast carcinoma and these changes show that there is an increase in the initiation of translation of the polypeptide chains on the ribosomes in the treated versus control human breast carcinoma. These changes in gene expression of serine and glycine biosynthesis and initiation of translation could be sparked by the significant increase in chloride ion channel expression that is a by-product of the _{b}Cl⁻ influence on diamagnetic anisotrophy in the cell membranes.

CLIC2 and CLIC4 code for chloride intracellular channel proteins and are members of the p64 family that bind to dynamin, tubulin, actin, and creatine kinase and were found to be upregulated by microarray analyses and subsequently validated by RT-qPCR (Suh, 2012; Cromer, 2007; Peretti et al., 2015) (Table 4). These families of ion channels are known to influence chloride transport, signaling, cytoskeleton integrity, mitosis, cytokinesis, and differentiation functions etc., of many tissues (Cromer, 2007; Peretti et al., 2015). CLIC2 and CLIC4 were upregulated 4-fold in RT-qPCR in the human breast carcinoma and have also been shown to participate in suppression of tumor growth. The absence of detectable levels of CLIC4 has been found to contribute to TGF-B resistance and enhance tumor development. Also, CLIC4 codes for a diverse group of proteins that regulates cellular processes, such as stabilization of cell Vmem (Peretti et al., 2015). No significant up-regulation of these 2 chloride channels expressions were found in the MCF-10A cells although expression was noted in both groups.

TABLE 4
Table 4. Unpaired t-tests of RT-qPCR for CLIC2 and CLIC4
Gene Expression. (Delta-Delta CT Methods showed 4-fold
Increase in CLIC2 and CLIC4 Gene Expression).

| | Unpaired t-tests | | | | |
|---|---|---|---|---|---|
| Gene | df | ts | p | Mean/Control | Mean/Treated |

MDA-MB231

| | | | | | |
|---|---|---|---|---|---|
| CLIC2 | 8 | 8.75038 | 0.0000228 | 11.23 | 9.53 |
| CLIC4 | 8 | 11.0548 | 0.000004 | 5.17264 | 3.106 |

Unpaired t-tests of RT-qPCR for CLIC2 and CLIC4 Gene Expression. (Delta-Delta CT Methods showed 4-fold Increase in CLIC2 and CLIC4 Gene Expression).

### Discussion

Chloride is a diamagnetic metal ion that appears to dissociate from its chloro-metabolites after exposure to the DC-DEP force EMF. The frequency emitted by this device appears to create a _{b}Cl⁻ that shows a strong affinity to the cellulose acetate filter that consists of properties similar to cell membranes, as shown in FIG. 4. The cell membrane exhibits a distinct diamagnetic anisotropy (Iwasaka et al., 2007). Diamagnetic anisotropy occurs when a field operates in opposition to the applied field and is driven by that applied field. This data shows that the diamagnetic negatively charged nature of _{b}Cl⁻ is attracted to the diamagnetic hydrophilic domain (like attracts like) of the filter and most likely the cell membrane possibly causing the spin states and fields from the _{b}Cl⁻ and water to operate in opposition to the positively charged spin states and fields of the paramagnetic, ferromagnetic and anti-ferromagnetic ions. Chemical shifts involve diamagnetic anisotropy or opposing bi-directional flow of ions and molecules in the membranes of cells. This applied DC-DEP force EMF discussed here shows that this carefully designed frequency disassociates and magnetizes the _{b}Cl⁻ from its chloro-metabolites and, through magnetic separation/attraction/repulsion mechanisms, drive the ion flow through the membranes without need for a cellular expenditure of ATP/GTPs. See FIGS. 1 & 4. The ferromagnetism displayed by the nickel could explain how the applied field continues to exert DC-DEP force EMF in solution before waning after approximately 90 hours as seen in the growth inhibitory effects in FIG. 2D.

The data show that this _{b}Cl⁻ also begins to diamagnetically influence not only the extracellular matrix and cell membrane but also the intracellular organelles through up-regulation of CLIC2, CLIC4, mRNA and tRNA in the human breast carcinoma (Table 4). Since chloride ion channels are known to play a prominent role in Vmem regulation and cell volume, the hyperpolarization noted by the Vmem assays and the significant cell volume differences also show chloride channel influence. The microarray data showed no significant change in other ion channel gene expressions such as Na⁺, K⁺-ATPase alpha and beta subunits and Ca⁺ channels in the human breast carcinoma or the human breast epithelial cells. Chloride ions are known to bind to tubulin and the tubulin fluorescent assays and phase contrast pictures both show distinct changes in cell morphology and mitosis after one day of growth in DC-DEP force EMF exposed growth media, as shown in FIG. 5 and FIG. 9.

Vmem in cancerous, injured and proliferating cells is ˜<−30 mV, while noncancerous cells have a resting potential of ˜>−70 mV on average (Zhou and Uesaka, 2006; Yang and Brackenbury, 2013; Levin, 2014; Lobikin et al., 2012). Cells have been found to respond to different electromagnetic signals when undergoing division, migration and differentiation and involve the changing gradients of Vmem and ion channel activity that are produced and sensed by both excitable and non-excitable cells (Yang and Brakenbury, 2013; McCaig, 2009; Funk et al., 2009). These data show that the magnetic behavioral changes in the _{b}Cl⁻ modulate chloride ion channels in human breast carcinoma leading to Vmem changes, growth inhibition and mitotic arrest. The microarray data and RT-qPCR validations show that endoplasmic reticulum stress is significantly up-regulated in the human breast carcinoma while this stress pathway is significantly down-regulated in the human breast epithelial cells. Since chloride ion channel is intricately linked to membrane potential regulation and cancerous cells are known to exist in a depolarized state with an out of control growth rate, the ability to up-regulate chloride ion channel expression is an elusive non-harmful mechanism that selectively targets cancer and also is linked to other co-morbidities. This DC-DEP force EMF frequency is the long awaited switch that transforms naturally occurring aqueous chloride that often stabilizes cations in the environment to _{b}Cl⁻ that stabilizes cell membranes, intracellular organelles, and ultimately cell physiology.

### CONCLUSION

The cell membranes have been described as liquid crystal semiconductors and these voltage gated chloride channels that have intrigued scientists in recent years may give credibility to this concept (Lipton, 2008). Due to space constraints, I have only discussed a few of the 2,468 significant changes in gene expression (2-26 fold increases) that were found in my research on these two cell lines. Preliminary analyses of the strong transcriptional reprogramming of these cells show that these changes appear to make biological sense in their connections to multiple targets and pathways. Experiments were also conducted with murine melanoma, human melanoma, HeLa, and human osteosarcoma cells that have also shown significant growth inhibition when cultured in the DC-DEP force EMF treated media while murine fibroblasts showed no growth inhibition in the disclosed experiments.

When a patient presents with imbalances in serum chloride levels, the only way to currently normalize this biomarker is by addressing hydration, renal, and endocrine issues. The ability to dielectrophoretically disassociate the _{b}Cl⁻ from its chloro-metabolites and influence chloride channel expression may open a door to the advancement in the understanding of both cell biology and ultimately clinical disease management.

### Supplemental Data

Microarray data, Agilent data and RT-qPCR data have been uploaded to NCBI GEO database, accession #GSE92320, which is incorporated in its entirety herein by reference.

Biochloride Generation and Methods: A Dielectrophoretic Electromagnetic Field Alteration of Living Cells

In an embodiment of the invention, the Bio-Field Array creates a direct current (dc) driven dielectrophoretic (DEP) electromagnetic field in a hypotonic saline solution. This dc-DEP-EMF changes the polarity of the ferroelectric chloride ion in an organism. This change in the polarity of the chloride ion facilitates anionic and cationic movement through the cell membranes primarily through the voltage-gated chloride ion channels (Purnell & Skrinjar, 2016). This BFA's dc-DEP-EMF can be applied directly to cells in culture by treating the saline solution for 30 minutes and then reconstituting cell media components with this solution prior to plating cells in this ‘treated’ media. It can also be applied to organisms (i.e., people, animals) by placing them in a water bath while the ˜2.5-3.0 amperes of dc current is applied to the “array” in the hypotonic saline solution while the organism has any, part or most of its body in the solution during the active field application for 30-35 minutes. Plants can also be watered with this dc DEP EMF treated saline solution for enhanced growth and disease resistance. Lastly, this BFA dc-DEP-EMF saline solution can also be infused intravenously into an organism in order to modulate chloride ion channels in the cells membranes of the organism. Chloride ion channel modulation is a novel application that has the potential to affect clinical disease management for diseases that include but are not limited to: cancer, neurodegeneration, inflammation, arthritis, cystic fibrosis, macular degeneration, myotonia, hypertension, polycystic kidney disease, epilepsy, sepsis etc. (Verkman, Levin, 2013; Purnell & Skrinjar, 2016)

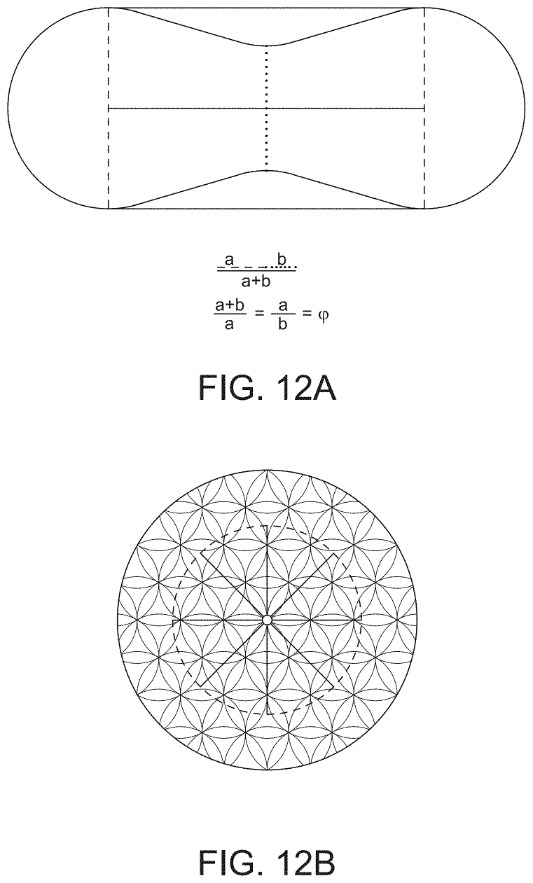

While we have observed chloride ion channel modulation (through ferroelectric changes in the polarity of the diamagnetic chloride ion) in eukaryotic cancerous and noncancerous cells in vitro, we have also now observed a reversal of rheological alterations in the human erythrocyte in our first safety and feasibility human study. Rheological alterations of red blood cells have been linked to many chronic diseases in humans (Ahmad & El-Sayed, 2003; Babu & Singh, 2004; Baskurt & Meiselman, 2003, 2010; Buttari et al., 2015; Ertan et al., 2017; Forsyth et al., 2012; Hierso et al., 2014; Hung et al., 1991; Kim et al., 2015; Meiselman et al., 2007; Piagnerelli et al., 2003; Rauf, 2013; Serroukh et al., 2017; Wang, 1993). The human erythrocyte must maintain a distinct and efficient biconcave discoid shape in order to efficiently deliver oxygen (O²) molecules and pick up carbon dioxide (CO²) molecules to be processed into H⁺/HCO3⁻ or to be expelled by the lungs (Vince et al., 2000). The erythrocyte functions much like a capacitor in solid-state electronics (Ho, Jow & Boggs, 2010). A capacitor stores energy as a dielectric constant (ε) between positively and negatively charged plates, whereas erythrocytes maintain their zeta potential (Z) as a dielectric constant (ε) between their negatively charged membrane surface and the positively charged adjacent Stern layer. The Golden Ratio, a function of Phi φ, offers a mathematical measure of the distinct and desired toroidal curvature of the red blood cell that is governed by this zeta potential (Livio, 2008; Zhang et al., 2016). We disclose a Golden Ratio proportion that could be used to measure the optimal proportions that are required for the optimal efficiency of the human erythrocyte, as shown in FIG. 12A-12B. These figures demonstrate the potential difference between the cation and anion distribution among the surface membrane and stern layers in a non-polarized RBC and a polarized RBC after exposure to the bio-field array sessions. Note separation in the anions and cations on the surface membrane of the red blood cell and the Stern Layer (like attracts like) in the polarized RBC. The RBC membrane function appears to be greatly affected by the immediate area surrounding the membrane surface. The separation of charge or the dielectrophoretic effect of the BFA allows for the free chloride/bicarbonate anion flow at the membrane level which remains separated from the positively charged Stern Layer. This zeta potential enhancement returns the red blood cell geometric Golden Ratio proportions that are optimal for CO2 neutralization/conversion to H⁺/HCO3⁻ where the H⁺ goes on to form hemoglobin to carry oxygen and the HCO3− goes on to facilitate pH regulation of the organism.

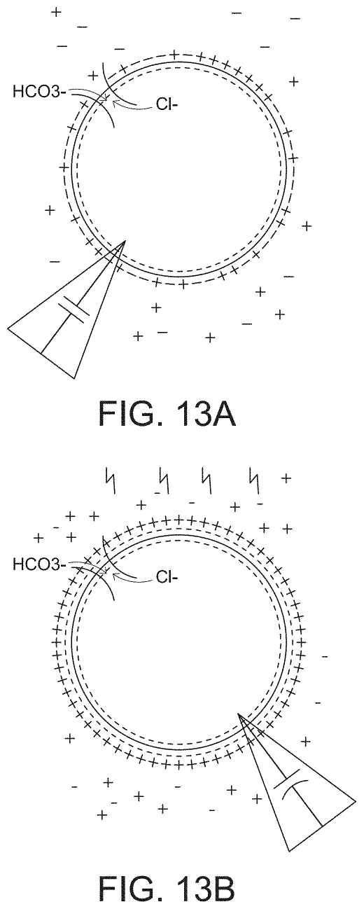

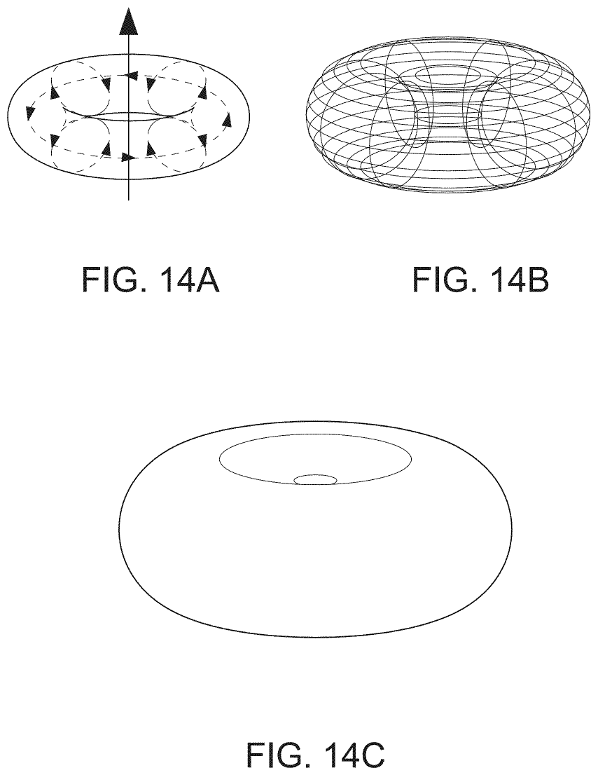

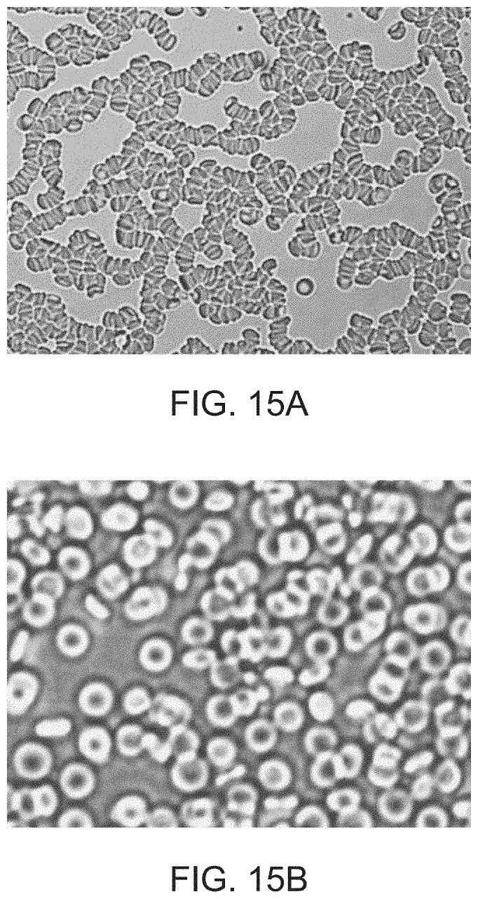

The anion transport capacity of the erythrocyte membrane is one of the largest of any cell membrane (Wieth, Andersen, Brahm, Bjerrum & Borders, 1982). The BFA-dc-DEP-EMF drives the zeta potential through a toroidal excitation of Band3/AE1 which is a chloride ion anion channel (whereas most other cell membranes are driven my magnetic multipoles the RBC is driven by a toroidal multipole), as shown in FIG. 14. Chloride must reside immediately adjacent to the surface membrane on the red blood cell since it is the gatekeeper of Band3/AE1, as shown in FIG. 13A-13B. When CO2 enters the red blood cell through Band 3/AE1 channel, a chloride ion must exit the cell to maintain cell neutrality. When HCO3− exits the red blood cell, a chloride ion must enter the red blood cell to retain cell neutrality. Therefore, maintaining separation between the negative surface membrane (where the chloride resides) and the positively charge cationic Stern layer is essential to maintain a strong zeta potential or a static current flow on the surface of the torus/red blood cell, as shown in FIG. 13A-13B (Papasimakis, Fedotov, Savinov, Raybould & Zheludev, 2016). Interestingly, the study participants in our feasibility pilot study also showed resolution of rheological changes in the red blood cells (via live blood analyses) and a significant decrease in serum CO² (p=0.017) over time which could correlate with an increased efficiency in the Band3/AE1 exchange and red blood cell function. (The study participants also showed a significant decrease/normalization of systolic and diastolic blood pressure over time as well as significant improvement of overall health via PROMIS questionnaires). The BFA's dc DEP EMF appears to enhance zeta potential thereby ensuring the appropriate geometric proportions that form Golden Ratio of the red blood cell and that are necessary for the electromagnetic excitation driven toroidal multipole flow, as shown in FIG. 14A, that ultimately maintains the efficiency of the center dielectrophoretic field, as shown in FIG. 14B and FIG. 15. This center DEP field is necessary for the dielectrophoretic separation of CO₂ and H₂O to occur which then in turn yields H⁺ that goes on to form hemoglobin (to carry O2) and HCO3⁻ that exits the red blood cell through Band 3/AE1 to assist with pH balance in the body. These geometric proportions of the Golden Ratio in the human erythrocyte could be measured in real time and ultimately used as a new biomarker for treatment of many chronic diseases that are known to be linked to rheological alterations and a distortion in the Golden Ratio of the RBC. (Liao et al., 2013). As shown in FIG. 15, red blood cells treated with BFA, return the geoemetric proportions of the Golden Ratio (1.618)

$$ϕ = \frac{( 1 + \sqrt{5} )}{2} = 1.6180339887 …$$

The ability to modulate chloride ions and chloride ion channels has remained elusive to date; it appears that the ability to alter the magnetic behavior of the chloride ion in all cell membranes with the BFA DC DEP EMF could be a new healthcare intervention. Now, the newly discovered BFA's DC DEP EMF ability to modulate the zeta potential through ferromagnetic, ferroelectric and ferroelastic changes in order to restore the designed Golden Ratio of the red blood cell could offer a new way to treat red blood cell rheology and ultimately cell, tissue and organism pathology with a novel dielectrophoretic electromagnetic application (Giovanna, 2014, Liao, Chang & Chang, 2013; Wang & Popel, 1993).

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. A solution containing biochloride as substantially described and disclosed herein.

2. A method of generating biochloride as substantially described and disclosed herein.

3. A method of treating cancer cells by contacting said cancer cells with a solution containing biochloride.

4. A method of modulating chloride ions and chloride ion channels using dielectrophoretic electromagnetic application to treat red blood cell rheology and cell, tissue and organism pathology.
